(19) United States
(12) Patent Application Publication
(10) Pub. No.: US 2022/0379103 A1
(43) Pub. Date: Dec. 1, 2022

(54) RETAINER ELEMENT TO SECURE A CATHETER ADAPTER VALVE

(71) Applicant: Becton, Dickinson and Company, Franklin Lakes, NJ (US)

(72) Inventors: Xiwei Chen, Shanghai (CN); Tao Jiang, Shanghai (CN); Yiping Ma, Layton, UT (US); Linda Kunardi, Singapore (SG); Kiat Jin Cheng, Singapore (SG); Thomson Wong, Singapore (SG)

(21) Appl. No.: 17/746,656

(22) Filed: May 17, 2022

Related U.S. Application Data
(60) Provisional application No. 63/194,060, filed on May 27, 2021.

(51) Int. Cl. A61M 39/02; A61M 25/00

(52) U.S. Cl. CPC A61M 39/0247; A61M 25/0097; A61M 2039/1077

(57) ABSTRACT

A catheter assembly may include a catheter adapter, which may include a distal end, a proximal end, a lumen extending through the distal end of the catheter adapter and the proximal end of the catheter adapter, and a side port disposed between the distal end of the catheter adapter and the proximal end of the catheter adapter. The catheter assembly may include a catheter extending distally from the distal end of the catheter adapter. The catheter assembly may include a valve disposed within the lumen. The catheter assembly may include a retainer element configured to secure the annular valve within the lumen. The retainer element may be disposed within the lumen proximal to a proximal end of the annular valve.

### RELATED APPLICATIONS

This application claims the benefit of U.S. Provisional Patent Application No. 63/194,060, filed on May 27, 2021, entitled RETAINER ELEMENT TO SECURE A CATHETER ADAPTER VALVE, which is incorporated herein in its entirety.

### BACKGROUND

Catheters are commonly used for a variety of infusion therapies. Catheters may be used for infusing normal saline solution, various medicaments, total parenteral nutrition, or other fluids into a patient. Catheters may also be used to withdraw blood from the patient for diagnostic or other purposes.

A common type of catheter is a peripheral intravenous catheter (“PIVC”) that is “over-the-needle.” As its name implies, the PIVC that is over-the-needle may be mounted over an introducer needle having a sharp distal tip. The PIVC and the introducer needle may be assembled so that the distal tip of the introducer needle extends beyond the distal tip of the PIVC with the bevel of the needle facing up away from skin of the patient. The PIVC and introducer needle are generally inserted at a shallow angle through the skin into the vasculature of the patient.

In order to verify proper placement of the introducer needle and/or the PIVC in the blood vessel, a clinician generally confirms that there is “flashback” of blood in a flashback chamber of the catheter assembly. Once placement of the needle has been confirmed, the clinician may remove the introducer needle, leaving the PIVC in place for future fluid infusion.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices and related systems and methods. More specifically, the present disclosure relates to retainer elements to secure a valve within a catheter adapter, as well as related devices, systems, and methods. In some embodiments, a catheter assembly may include a catheter adapter, which may include a distal end, a proximal end, and a lumen extending through the distal end of the catheter adapter and the proximal end of the catheter adapter. In some embodiments, the catheter adapter may include a side port disposed between the distal end of the catheter adapter and the proximal end of the catheter adapter.

In some embodiments, the catheter assembly may include a catheter secured within the catheter adapter and extending distally from the distal end of the catheter adapter. In some embodiments, the catheter assembly may include an annular valve disposed within the lumen and configured to seal the side port from the lumen. In some embodiments, the catheter assembly may include a retainer element configured to secure the annular valve within the lumen. In some embodiments, the retainer element may be disposed within the lumen proximal to a proximal end of the annular valve or within the proximal end of the annular valve. In some embodiments, the retainer element may contact the annular valve.

In some embodiments, the retainer element may include a ring, and the ring may be disposed proximal to the annular valve. In some embodiments, the retainer element may include a C-shape, and the retainer element may pinch the annular valve between the C-shape and the catheter adapter. In some embodiments, a portion of the annular valve within an open portion of the C-shape may be unclamped, which may facilitate opening of the annular valve in response to infusion of fluid from the side port into the lumen. In some embodiments, the portion of the annular valve may be on a same side of the catheter assembly as the side port. In some embodiments, the catheter assembly may include an antimicrobial coating disposed on an outer surface of the retainer element.

In some embodiments, the catheter assembly may include a valve disposed within the lumen, and a C-shaped retainer element secured within the lumen proximal to the valve. In some embodiments, an inner surface of the catheter adapter may form the lumen and may include a narrowed diameter portion proximal to the valve. In some embodiments, the C-shaped retainer element may be configured to compress to move distally through the narrowed diameter portion. In some embodiments, the C-shaped retainer element may be proximate the valve.

In some embodiments, after moving distally through the narrowed diameter portion, the C-shaped retainer element may be configured to expand within the lumen proximate the valve. In some embodiments, the lumen may include a first inner diameter distal to the narrowed diameter portion and proximal to the valve and a second inner diameter at the narrowed diameter portion. In some embodiments, the second inner diameter may be less than the first inner diameter. In some embodiments, the lumen may include a third inner diameter proximal to the narrowed diameter portion. In some embodiments, the second inner diameter may be less than the third inner diameter.

In some embodiments, a width of the C-shaped retainer element may be greater than a distance from the narrowed diameter portion to the proximal end of the catheter adapter. In some embodiments, the first inner diameter may be equal to the second inner diameter. In some embodiments, an outer diameter of the C-shaped retainer element in a natural state may be greater than the first inner diameter.

In some embodiments, in the natural state, a distal end of the C-shaped retainer element may include a first outer diameter and a proximal end of the C-shaped retainer element may include a second outer diameter, and the second outer diameter may be greater than the first outer diameter. In some embodiments, an inner surface of the catheter adapter forms the lumen, wherein the first outer diameter and the second outer diameter contacts the inner surface.

In some embodiments, the catheter assembly may include the retainer element, which may be configured to secure the annular valve within the lumen, and the retainer element is disposed within the lumen proximal to a proximal end of the annular valve. In some embodiments, the retainer element may include a spring or a metal disc. In some embodiments, the spring may be tapered inwardly in a proximal direction. In some embodiments, the metal disc may include a central opening. In some embodiments, an outer edge of the metal disc may include multiple proximally-extending protrusions configured to press into the inner surface. In some embodiments, an edge of the opening may include multiple distally-extending teeth.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of a prior art catheter assembly;

FIG. 1B is an upper perspective view of a prior art annular valve;

FIG. 1C is a cross-sectional view of the prior art catheter assembly;

FIG. 2A is a cross-sectional view of the prior art catheter assembly, illustrating the annular valve in an open position;

FIG. 2B is a cross-sectional view of the prior art catheter assembly, illustrating a distal end and a proximal end of the annular valve deformed;

FIG. 3A is a cross-sectional view of an example catheter assembly, illustrating an example retainer element, according to some embodiments;

FIG. 3B is an enlarged cross-sectional view of a portion of the catheter assembly of FIG. 3A, according to some embodiments;

FIG. 3C is an upper perspective view of the retainer element of FIG. 3A, according to some embodiments;

FIG. 3D is a cross-sectional view of a proximal end of the catheter assembly of FIG. 3A, according to some embodiments;

FIG. 4A is a cross-sectional view of an example catheter assembly, illustrating an example retainer element, according to some embodiments;

FIG. 4B is an upper perspective view of the retainer element of FIG. 4A, according to some embodiments;

FIG. 4C is a cross-sectional view of a proximal end of the catheter assembly of FIG. 4A, according to some embodiments;

FIG. 5A is a cross-sectional view of an example catheter assembly, according to some embodiments;

FIG. 5B is a perspective view of an example retainer element, illustrating an example retainer element in a natural state or uncompressed, according to some embodiments;

FIG. 5C is an upper perspective view of the retainer element of FIG. 5B, illustrating the retainer element in the natural state or uncompressed, according to some embodiments;

FIG. 6A is a partial cutaway view of the catheter assembly of FIG. 5A, illustrating the retainer element of FIG. 5B being inserted into an example catheter adapter, according to some embodiments;

FIG. 6B is an upper perspective view of the retainer element of FIG. 5B, illustrating the retainer element of FIG. 5B in a compressed state, according to some embodiments;

FIG. 6C is a partial cutaway view of the catheter assembly of FIG. 5A, illustrating the retainer element inserted into the catheter adapter, according to some embodiments;

FIG. 6D is a partial cutaway view of an example catheter assembly, illustrating an example retainer element, according to some embodiments;

FIG. 6E is a partial cutaway view of an example catheter assembly, illustrating an example retainer element that includes an antibacterial coating, according to some embodiments;

FIG. 7A is an upper perspective view of an example catheter assembly, according to some embodiments;

FIG. 7B is an upper perspective view of an example retainer element, according to some embodiments;

FIG. 7C is a cross-sectional view of the retainer element of FIG. 7B, according to some embodiments;

FIG. 7D is a partial cutaway view of the catheter assembly of FIG. 7A, illustrating the retainer element of FIG. 7B, according to some embodiments;

FIG. 8A is an upper perspective view of an example catheter assembly, illustrating an example retainer element, according to some embodiments;

FIG. 8B is an upper perspective view of the retainer element of FIG. 8A, according to some embodiments;

FIG. 8C is a side view of the retainer element of FIG. 8A, according to some embodiments;

FIG. 8D is an enlarged partial cutaway view of the catheter assembly of FIG. 8A, according to some embodiments;

FIG. 8E is an upper perspective view of an example retainer element, according to some embodiments; and

FIG. 8F is a side view of the retainer element of FIG. 8E, according to some embodiments.

### DESCRIPTION OF EMBODIMENTS

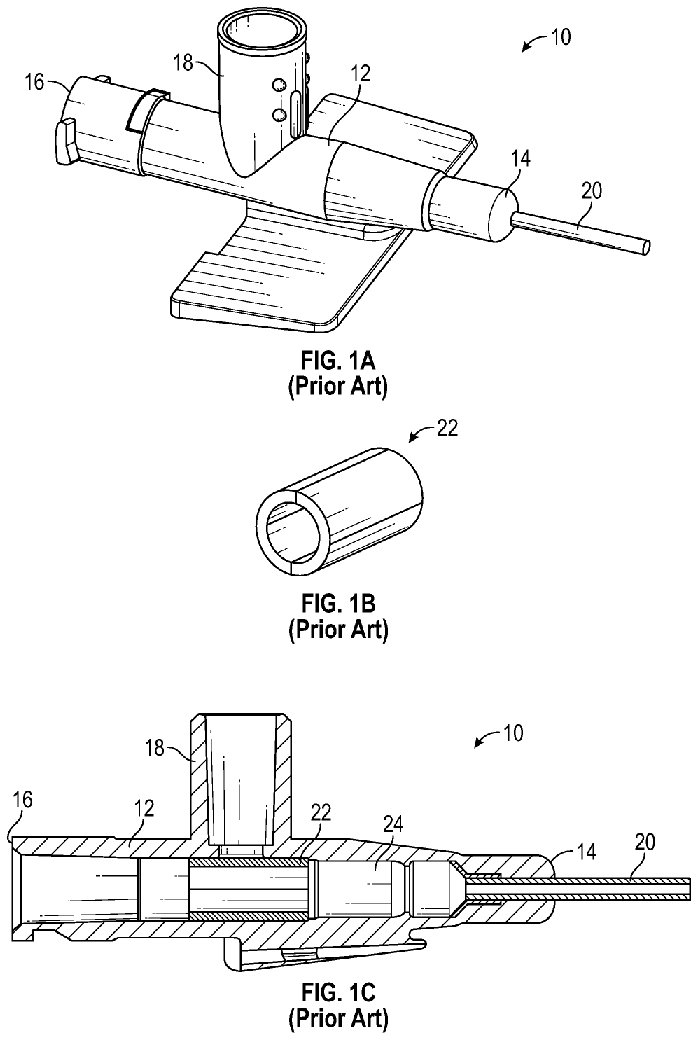

Referring now to FIGS. 1A-1C, a prior art catheter assembly 10 is illustrated. The prior art catheter assembly 10 includes a catheter adapter 12, which includes a distal end 14 and a proximal end 16. A side port 18 is disposed between the distal end 14 and the proximal end 16 and is used for injection of fluid and medications. A catheter 20 extends distally from the distal end 14 of the catheter adapter 12. The prior art catheter assembly 10 includes an annular valve 22 configured to seal the side port 18 from a lumen 24 of the catheter adapter that extends through the distal end 14 and the proximal end 16.

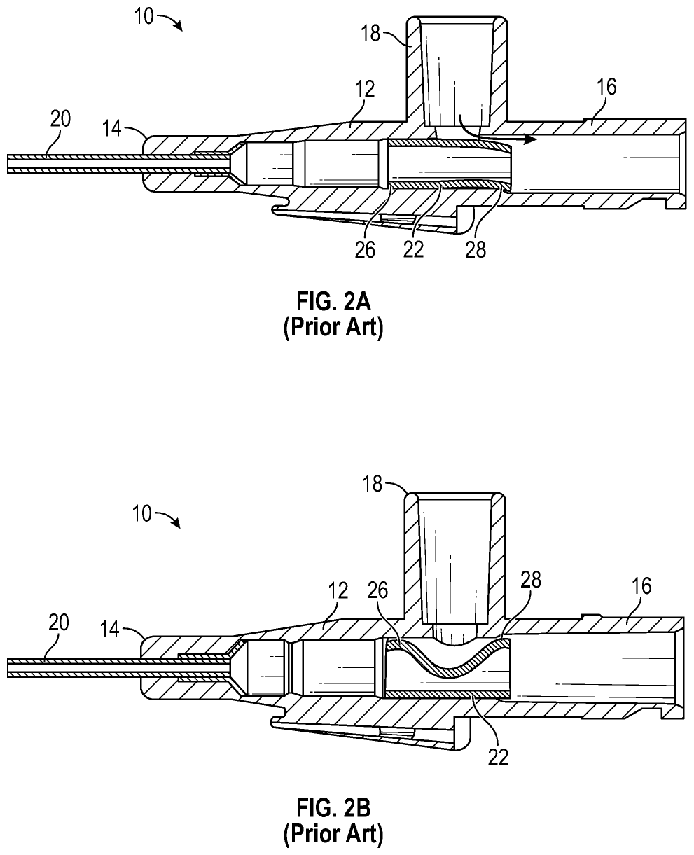

Referring now to FIG. 2A, a user may connect a syringe to the side port 18 and inject fluid into the side port 18. Pressure generated by the user pushing down on a plunger of the syringe and injecting fluid may deform a proximal end of the annular valve 22 downward to create a gap between the annular valve 22 and an inner surface of the catheter adapter 12. In response to deforming of the proximal end of the annular valve 22, fluid may then flow into the lumen 24.

Referring now to FIG. 2B, over repetitive usage of the annular valve 22, the fluid injected into the side port 18 tends to migrate into spaces between the annular valve 22 and the inner surface of the catheter adapter 12, leading to deformation of the annular valve 22 and reducing adhesion between the annular valve 22 and the catheter adapter 12. During subsequent injection of fluid, the annular valve 22 can be displaced a large distance in a proximal direction, leading to unsealing of the side port 18 and leakage of infusates or blood.

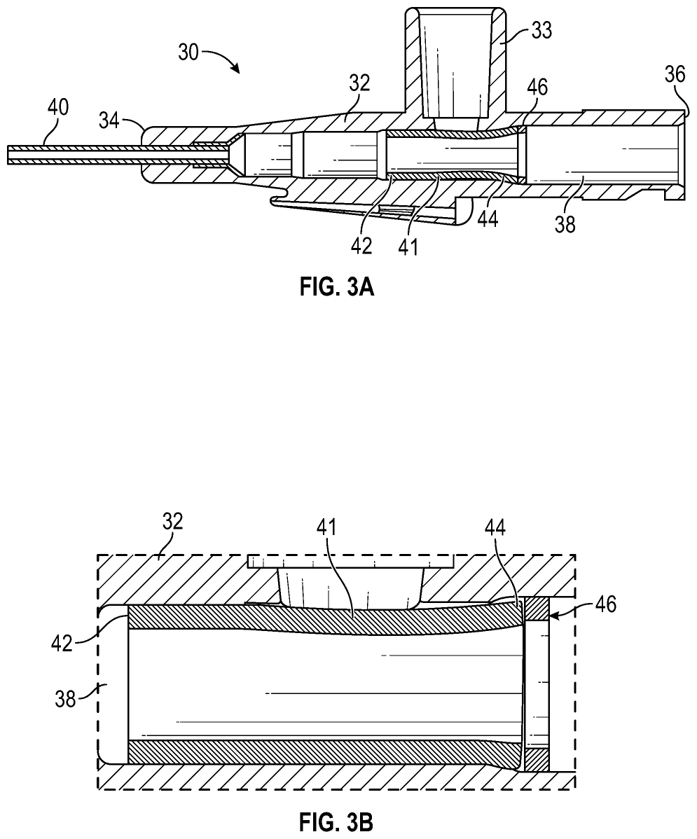

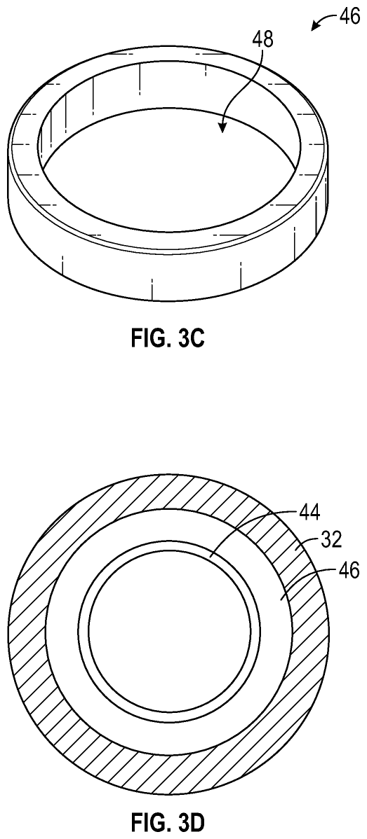

Referring now to FIGS. 3A-3D, in some embodiments, a catheter assembly 30 may include a catheter adapter 32, which may include a distal end 34, a proximal end 36, and a lumen 38 extending through the distal end 34 of the catheter adapter 32 and the proximal end 36 of the catheter adapter 32. In some embodiments, the catheter adapter 32 may include a side port 33 disposed between the distal end 34 of the catheter adapter 32 and the proximal end 36 of the catheter adapter 32. In some embodiments, the catheter assembly 30 may be similar or identical to the prior art catheter assembly 10 in terms of one or more features and/or operation.

In some embodiments, the catheter assembly 30 may include a catheter 40 secured within the catheter adapter 32 and extending distally from the distal end 34 of the catheter adapter 32. In some embodiments, the catheter 40 may include a peripheral intravenous catheter, a midline catheter, or a peripherally-inserted central catheter. In some embodiments, the catheter assembly 30 may include an annular valve 41 disposed within the lumen 38 and configured to seal the side port 33 from the lumen 38.

In some embodiments, the annular valve 41 may include a distal end 42 and a proximal end 44. In some embodiments, the catheter assembly 30 may include a retainer element 46 configured to secure the annular valve 41 within the lumen 38. In some embodiments, the retainer element 46 may be disposed within the lumen 38 proximal to the proximal end 44 of the annular valve 41. In some embodiments, the retainer element 46 may be in contact the annular valve 41. In some embodiments, the retainer element 46 may contact the annular valve 41 in response to infusion of fluid through the side port 33.

In some embodiments, the retainer element 46 may include a ring, and the ring may be disposed proximal to the annular valve 41. In some embodiments, the ring may be press-fit within the lumen 38 of the catheter adapter 32 or secured in another suitable manner. In some embodiments, the ring may prevent the annular valve 41 from moving proximally in response to injection of fluid through the side port 33, which may include a high-pressure injection equal to or greater than 300 psi. In some embodiments, a width of the retainer element 46 may be about 0.5 mm or another suitable width to reduce movement of the annular valve 41.

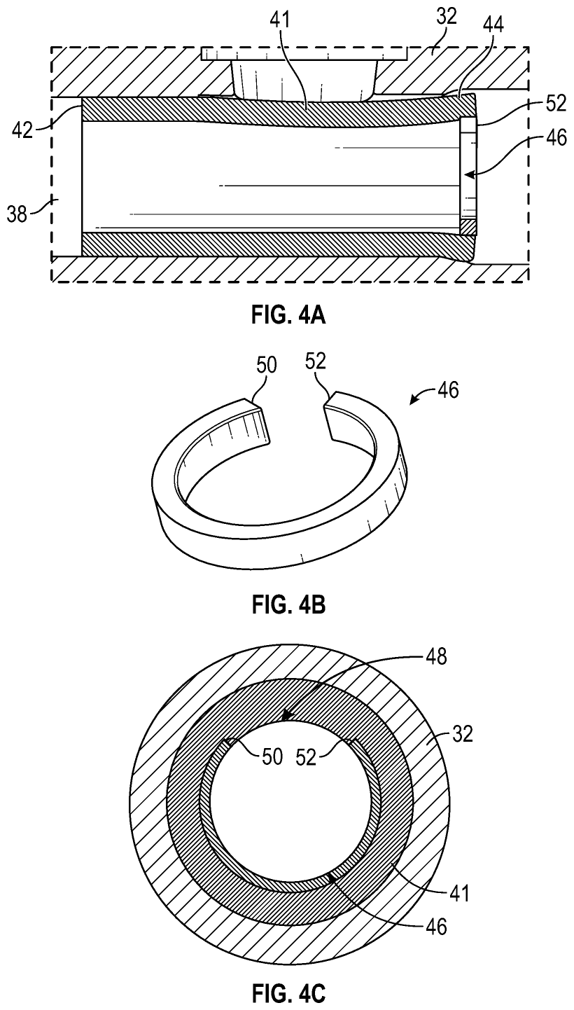

Referring now to FIGS. 4A-4C, in some embodiments, the retainer element 46 may include a C-shape, and the retainer element 46 may pinch or clamp the annular valve 41 between the C-shape and the catheter adapter 32. In some embodiments, a portion of the annular valve 41 within an open portion 48 of the C-shape may be unclamped, which may facilitate opening of the annular valve 41 in response to infusion of fluid from the side port 33 into the lumen 38. In some embodiments, the open portion 48 of the C-shape may be between a first end 50 and a second end 52 of the C-shape. In some embodiments, the portion of the annular valve 41 may be on a same side of the catheter assembly 30 as the side port 33. Thus, in some embodiments, the proximal end 44 of the annular valve 41 may deform downwardly to allow fluid to flow into the lumen 38 from the side port 33 during fluid infusion. In some embodiments, the side port 33 may be disposed on a top of the catheter adapter 32 and the portion may be disposed on a top of the annular valve 41. In some embodiments, the catheter assembly 30 may include an antimicrobial coating disposed on an outer surface of the retainer element 46.

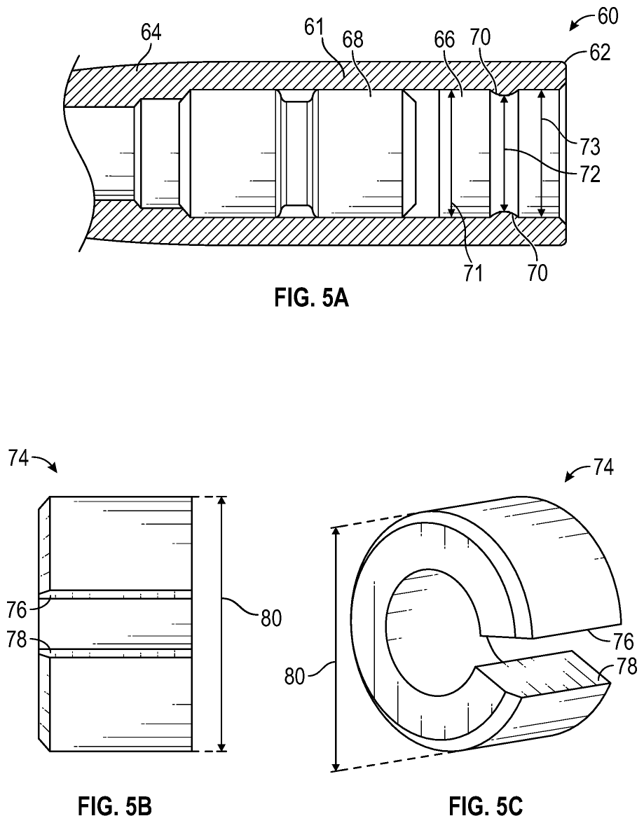

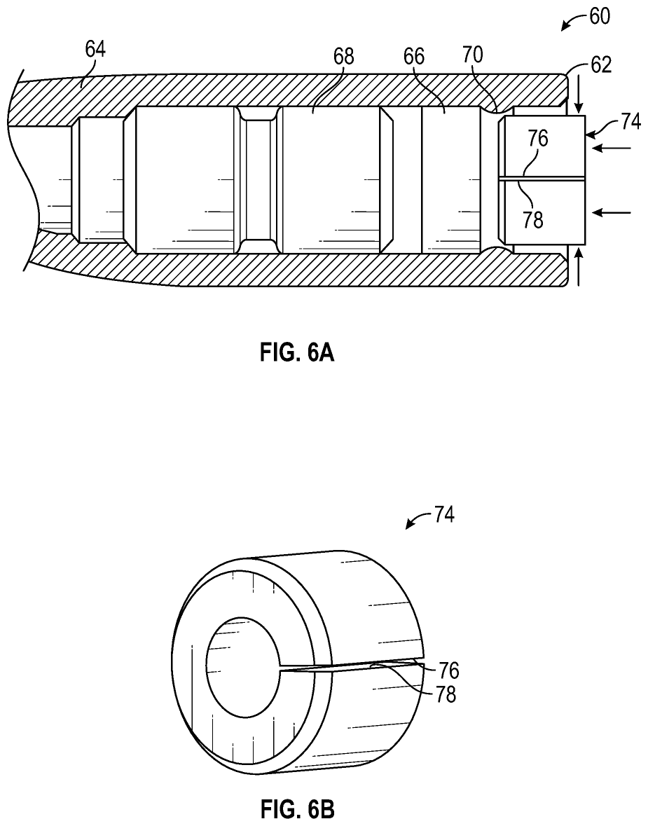

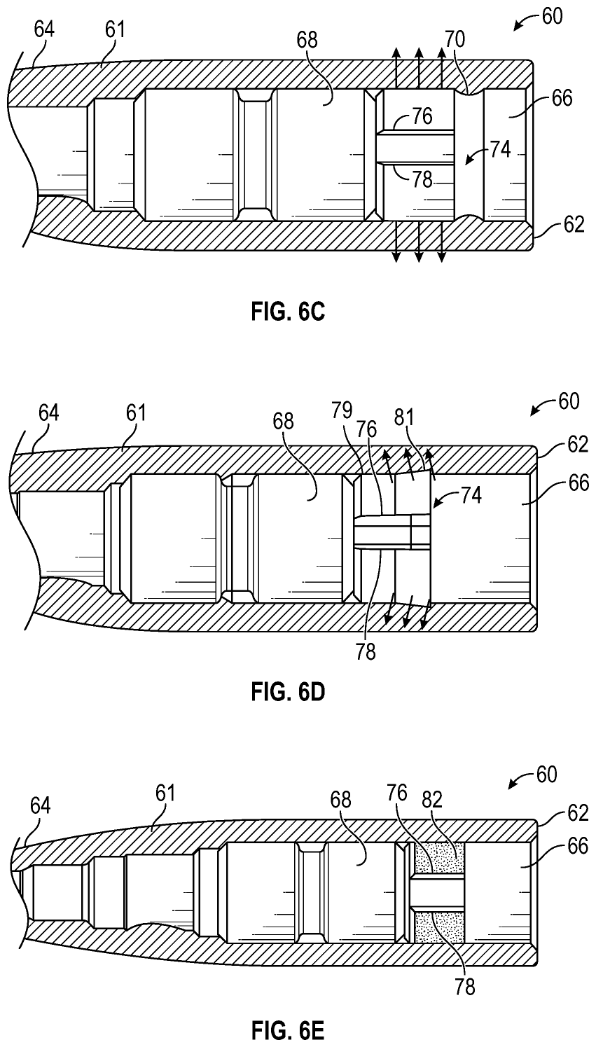

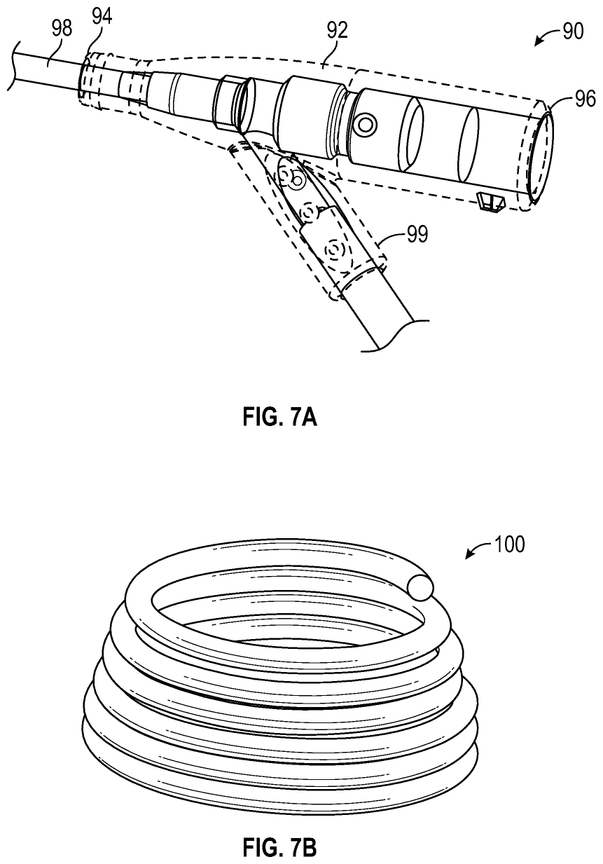

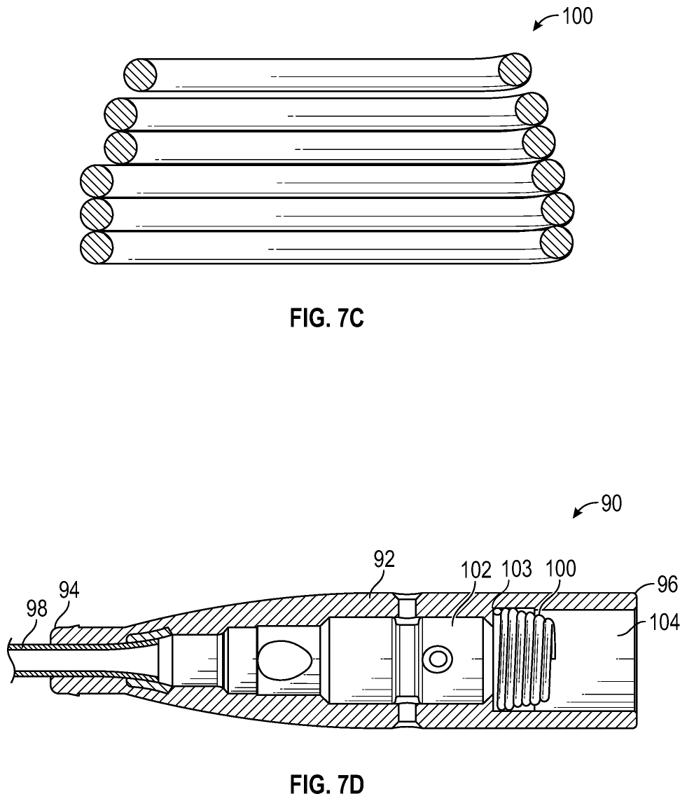

Referring now to FIGS. 5A-6C, in some embodiments, a catheter assembly 60 may include a catheter adapter 61, which may include a proximal end 62, a distal end 64, and a lumen 66 extending through the distal end 64 of the catheter adapter 61 and the proximal end 62 of the catheter adapter 61. In some embodiments, the catheter adapter 61 may include a side port (see for example, side port 99 in FIG. 7A) disposed between the distal end 64 of the catheter adapter 61 and the proximal end 62 of the catheter adapter 21. In some embodiments, the catheter assembly 60 may include a catheter secured within the catheter adapter 61 and extending distally from the distal end 64 of the catheter adapter 61. In some embodiments, a valve 68 disposed within the lumen 66 may include a one-piece septum, a two piece-septum, or another suitable septum or valve.

In some embodiments, an inner surface of the catheter adapter 61 may form the lumen 66 and may include a narrowed diameter portion 70 proximal to the valve 68. In some embodiments, the lumen 66 may include a first inner diameter 71 distal to the narrowed diameter portion 70 and proximal to the valve 68 and a second inner diameter 72 at the narrowed diameter portion 70. In some embodiments, the second inner diameter 72 may be less than the first inner diameter 71. In some embodiments, the lumen 66 may include a third inner diameter 73 proximal to the narrowed diameter portion 70. In some embodiments, the second inner diameter 72 may be less than or equal to the third inner diameter 73.

In some embodiments, the valve 68 within the lumen 66 may be configured to seal the proximal end 62 of the catheter adapter. In some embodiments, the catheter assembly 60 may include a C-shaped retainer element 74 configured to secure the valve 68 within the lumen 66. For example, the C-shaped retainer element 74 may secure the valve 68 in response to injection of fluid through the side port, which may include a high-pressure injection equal to or greater than 300 psi.

In some embodiments, the C-shaped retainer element 74 may be disposed within the lumen 66 proximal to the valve 68. In some embodiments, the C-shaped retainer element 74 may be proximate the valve 68. In some embodiments, the C-shaped retainer element 74 may be in contact the valve 68. In some embodiments, the C-shaped retainer element 74 may contact the valve in response to infusion of fluid through the side port distal to the valve 68.

It should be understood that the embodiments may be combined. For example, the catheter assembly 60 may be similar or identical to the catheter assembly 30 of FIGS. 3-4 and/or the prior art catheter assembly 10 in terms of one or more features and/or operation. In some embodiments, the C-shaped retainer element 74 may be disposed within the catheter adapter 32, which may include the narrowed diameter portion 70, and/or proximal to the annular valve 41. In some embodiments, the valve 68 may be replaced with the annular valve 41.

In some embodiments, the C-shaped retainer element 74 may be secured within the lumen 66 proximal to the valve 68. In some embodiments, the C-shaped retainer element 74 may be configured to compress to move distally through the narrowed diameter portion 70, as illustrated, for example, in FIGS. 6A-6B. In some embodiments, in response to the C-shaped retainer element 74 being compressed, a first end 76 and a second end 78 of the C-shaped retainer element 74 may be brought closer together and/or in contact.

In some embodiments, after moving distally through the narrowed diameter portion 70, the C-shaped retainer element 74 may be configured to expand within the lumen 66 distal to the narrowed diameter portion 70 and/or proximate the valve 68, as illustrated, for example, in FIG. 6C. In these and other embodiments, the C-shaped retainer element 74 may be engaged in a press-fit with the catheter adapter 61. In some embodiments, a width of the C-shaped retainer element 74 may be greater than a distance from the narrowed diameter portion 70 to the proximal end 62 of the catheter adapter 61, as illustrated, for example, in FIG. 6A. In some embodiments, the width of the C-shaped retainer element 74 greater than the distance from the narrowed diameter portion 70 to the proximal end 62 may facilitate insertion of the C-shaped retainer element 74 into the catheter adapter 61 by the user.

In some embodiments, the first inner diameter 71 may be equal to the second inner diameter 72. In some embodiments, an outer diameter 80 of the C-shaped retainer element 74 in a natural state, illustrated, for example, in FIGS. 5B-5C, may be greater than the first inner diameter 71, such that the C-shaped retainer element 74 is compressed to move distal to the narrowed diameter portion 70. In some embodiments, the narrowed diameter portion 70 may include a protrusion, two opposing protrusions, or an annular protrusion.

Referring now to FIG. 6D, an outer diameter of the C-shaped retainer element 74 may increase in a proximal direction. In some embodiments, in the natural state, a distal end 79 of the C-shaped retainer element 74 may include a first outer diameter and a proximal end 81 of the C-shaped retainer element 74 may include a second outer diameter. In some embodiments, the second outer diameter may be greater than the first outer diameter. In some embodiments, the second outer diameter and the proximal end 81 may include a barb, two opposing barbs, or an annular barb (as illustrated, for example, in FIG. 6D). In some embodiments, the second outer diameter may increase friction between the C-shaped retainer element 74 and the catheter adapter 61 to prevent shifting of the valve 68. In some embodiments, the inner surface of the catheter adapter 61 may form the lumen 66, and the first outer diameter and the second outer diameter may contact the inner surface. Referring now to FIG. 6F, in some embodiments, the catheter assembly 60 may include an antimicrobial coating 82 disposed on an outer surface of the C-shaped retainer element 74.

Referring now to FIGS. 7A-7D, in some embodiments, a catheter assembly 90 may include a catheter adapter 92, which may include a distal end 94, a proximal end 96, and a lumen extending through the distal end 94 and the proximal end 96. In some embodiments, a catheter 98 may extend distally from the distal end 94 of the catheter adapter 92. In some embodiments, the catheter assembly 90 may include a retainer element 100. In some embodiments, a valve 102 disposed within a lumen 104 extending through the distal end 94 and the proximal end 96 may include a one-piece septum, a two piece-septum, or another suitable septum or valve. In some embodiments, the catheter adapter 92 may include a side port 99 in fluid communication with the lumen distal to the valve 102.

In some embodiments, the catheter assembly 90 may be similar or identical in terms of one or more features and/or operation to one or more of the following: the catheter assembly 60 of FIGS. 4-5, the catheter assembly 30 of FIGS. 3-4, and the prior art catheter assembly 10. In some embodiments, the retainer element 100 or the retainer element 106 may be disposed within the catheter adapter 32, which may include the flange 103, and/or proximal to the annular valve 41. In some embodiments, the valve 102 may be replaced with the annular valve 41.

In some embodiments, the retainer element 100 may be configured to secure the valve 102 within the lumen 104, and the retainer element 100 may be disposed within the lumen 104 proximal to a proximal end of the valve 102. In some embodiments, the retainer element 100 may include a spring, as illustrated, for example, in FIGS. 7B-7D. In some embodiments, the spring may be tapered inwardly in a proximal direction, which may allow the spring to act as a wedge to hold the valve 102 if the valve 102 should get displaced by high pressure, thus maintaining a seal.

In some embodiments, the proximal end of the spring may contact a flange 103 of the catheter adapter 92. In some embodiments, the proximal end of the spring may be engaged in an interference fit with the catheter adapter 92. In some embodiments, the proximal end of the spring may be engaged in an interference fit with at the flange 103 and/or proximal to the flange 103. In some embodiments, movement of the valve 102 into the spring may cause a diameter of an end coil of the spring in contact with the catheter adapter 92 to expand, further increasing interference of the spring with the catheter adapter 92. In some embodiments, the retainer element 100 may prevent dislodgement of the valve 102 during infusion of fluid into the side port at high pressures, such as, for example, 300 psi.

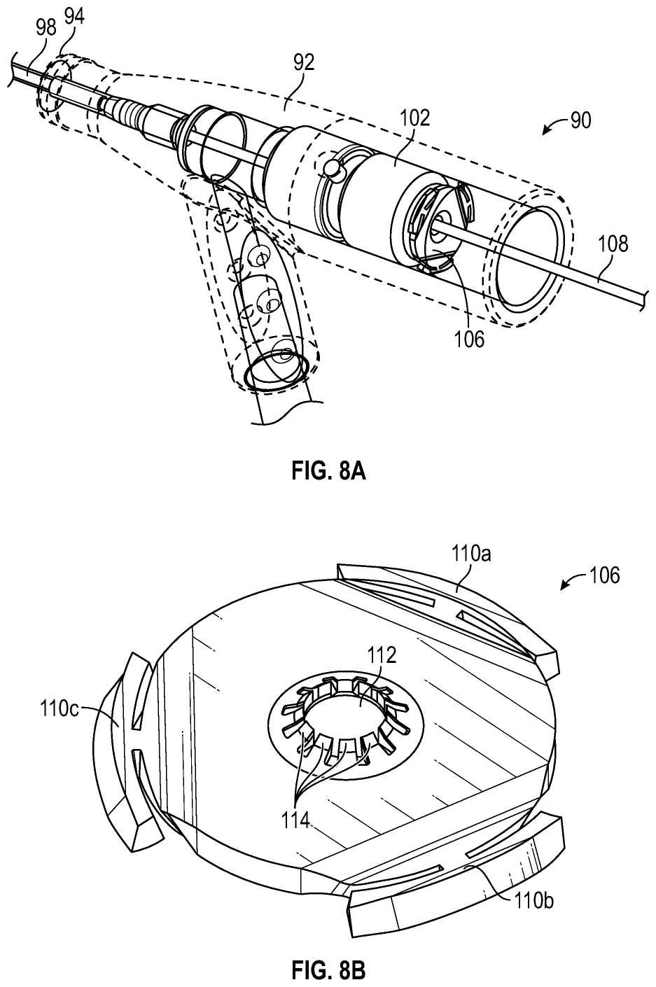

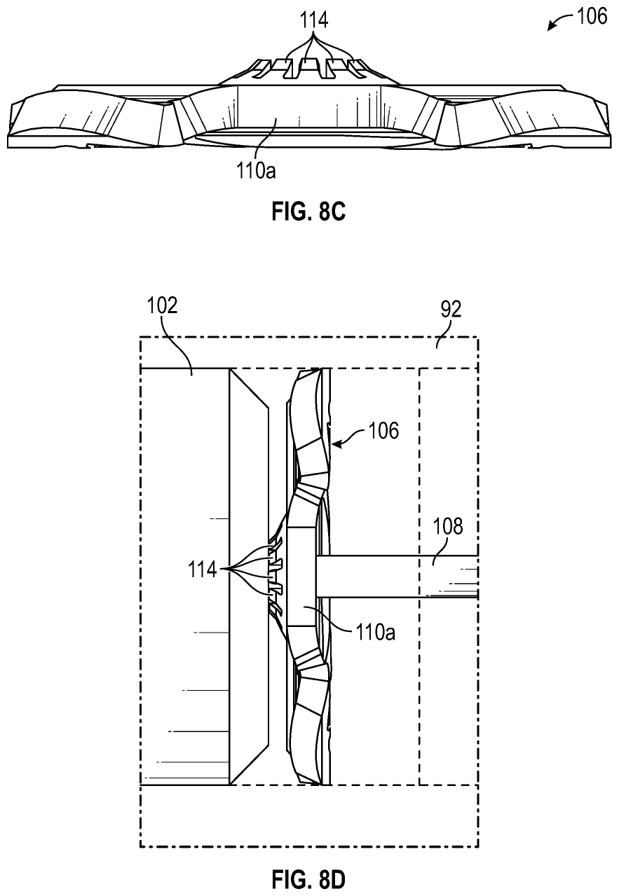

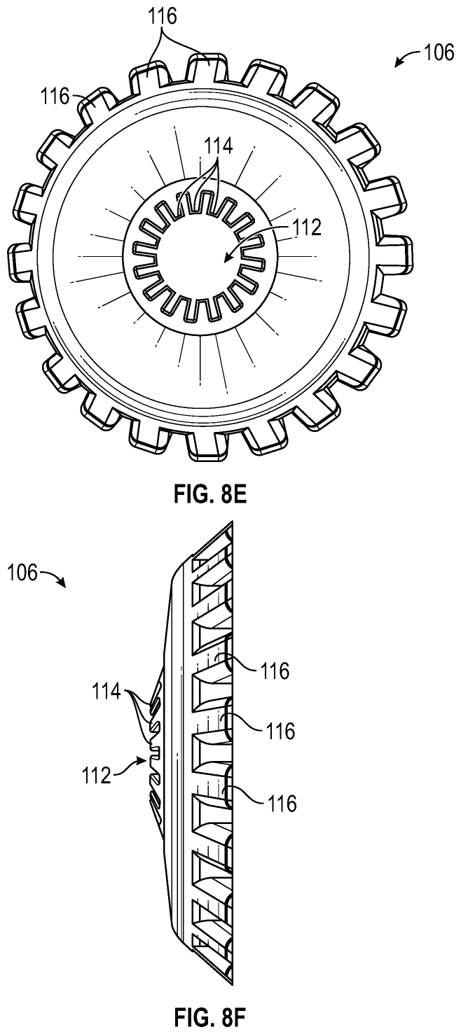

Referring now to FIGS. 8A-8F, in some embodiments, a retainer element 106 of the catheter assembly 90 may include a metal disc, which may be circular in shape. In some embodiments, the retainer element 106 may be positioned proximal to the valve 102 and may prevent the valve 102 from being ejected at high pressure, thus maintaining the seal.

As illustrated in FIGS. 8A-8D, in some embodiments, an outer edge of the metal disc may include multiple proximally-extending protrusions 110 configured to press into the inner surface of the catheter adapter 92. In some embodiments, the proximally-extending protrusions 110 may act as claws that poke into the inner surface of the catheter adapter 92. In some embodiments, the proximally-extending protrusions 110 may be evenly spaced around the outer edge of the metal disc. In some embodiments, the metal disc may include three proximally-extending protrusions 110, which may facilitate adequate securement of the valve 102, or another suitable number.

In some embodiments, the metal disc may include a central opening 112. In some embodiments, the central opening 112 may be just large enough for an introducer needle 108 to pass through. In some embodiments, an edge of the central opening 112 may include multiple distally-extending teeth 114, which may grip onto the valve 102, which may be elastomeric. Thus, in some embodiments, the distally-extending teeth 114 may reduce a likelihood of the valve 102 squeezing through the central opening 112 when high pressure is applied.

As illustrated in FIGS. 8E-8F, in some embodiments, the outer edge of the retainer element 106 may include multiple proximally-extending teeth 116. In some embodiments, the proximally-extending teeth 116 may act as claws that poke into the inner surface of the catheter adapter 92. In some embodiments, the proximally-extending teeth 116 may be evenly spaced around the outer edge of the metal disc. In some embodiments, a large number of the proximally-extending teeth 116 may increase a surface area in contact with the inner surface of the catheter adapter. For example, the metal disc may include 18-22 of the proximally-extending teeth 116. In some embodiments, the metal disc may include any suitable number of proximally-extending teeth 116.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter assembly, comprising:
a catheter adapter, comprising a distal end, a proximal end, a lumen extending through the distal end of the catheter adapter and the proximal end of the catheter adapter, and a side port disposed between the distal end of the catheter adapter and the proximal end of the catheter adapter;
a catheter extending distally from the distal end of the catheter adapter;
an annular valve disposed within the lumen and configured to seal the side port from the lumen; and
a retainer element configured to secure the annular valve within the lumen, wherein the retainer element is disposed within the lumen proximal to a proximal end of the annular valve or within the proximal end of the annular valve.

2. The catheter assembly of claim 1, wherein the retainer element contacts the annular valve.

3. The catheter assembly of claim 1, wherein the retainer element comprises a ring, wherein the ring is disposed proximal to the annular valve.

4. The catheter assembly of claim 1, wherein the retainer element comprises a C-shape, wherein the retainer element pinches the annular valve between the C-shape and the catheter adapter.

5. The catheter assembly of claim 4, wherein a portion of the annular valve within an open portion of the C-shape is unclamped.

6. The catheter assembly of claim 5, wherein the portion of the annular valve is on a same side of the catheter assembly as the side port.

7. The catheter assembly of claim 1, further comprising an antimicrobial coating disposed on an outer surface of the retainer element.

8. A catheter assembly, comprising:
a catheter adapter, comprising a distal end, a proximal end, and a lumen extending through the distal end of the catheter adapter and the proximal end of the catheter adapter;
a catheter extending distally from the distal end of the catheter adapter;
a valve disposed within the lumen; and
a C-shaped retainer element secured within the lumen proximal to the valve.

9. The catheter assembly of claim 8, wherein an inner surface of the catheter adapter forms the lumen and comprises a narrowed diameter portion proximal to the valve, wherein the C-shaped retainer element is configured to compress to move distally through the narrowed diameter portion.

10. The catheter assembly of claim 9, wherein the C-shaped retainer element is proximate the valve.

11. The catheter assembly of claim 10, wherein after moving distally through the narrowed diameter portion, the C-shaped retainer element is configured to expand within the lumen proximate the valve.

12. The catheter assembly of claim 9, wherein the lumen comprises a first inner diameter distal to the narrowed diameter portion and proximal to the valve and a second inner diameter at the narrowed diameter portion, wherein the second inner diameter is less than the first inner diameter.

13. The catheter assembly of claim 12, wherein the lumen further comprises a third inner diameter proximal to the narrowed diameter portion, wherein the second inner diameter is less than the third inner diameter.

14. The catheter assembly of claim 13, wherein a width of the C-shaped retainer element is greater than a distance from the narrowed diameter portion to the proximal end of the catheter adapter.

15. The catheter assembly of claim 13, wherein the first inner diameter is equal to the second inner diameter.

16. The catheter assembly of claim 12, wherein an outer diameter of the C-shaped retainer element in a natural state is greater than the first inner diameter.

17. The catheter assembly of claim 9, wherein in a natural state, a distal end of the C-shaped retainer element comprises a first outer diameter and a proximal end of the C-shaped retainer element comprises a second outer diameter, wherein the second outer diameter is greater than the first outer diameter, wherein an inner surface of the catheter adapter forms the lumen, wherein the first outer diameter and the second outer diameter contacts the inner surface.

18. A catheter assembly, comprising:
a catheter adapter, comprising a distal end, a proximal end, and a lumen extending through the distal end of the catheter adapter and the proximal end of the catheter adapter;
a catheter extending distally from the distal end of the catheter adapter;
a valve disposed within the lumen; and
a retainer element configured to secure the annular valve within the lumen, wherein the retainer element is disposed within the lumen proximal to a proximal end of the annular valve, wherein the retainer element comprises:
a spring; or
a metal disc.

19. The catheter assembly of claim 18, wherein the spring is tapered inwardly in a proximal direction.

20. The catheter assembly of claim 18, wherein the metal disc comprises a central opening, wherein an outer edge of the metal disc comprises a plurality of proximally-extending protrusions configured to press into the inner surface, wherein an edge of the opening comprises a plurality of distally-extending teeth.
